(19) United States
(12) Patent Application Publication
(10) Pub. No.: US 2025/0352780 A1
(43) Pub. Date: Nov. 20, 2025

(54) SELECTIVE BLOCKING DOOR DEVICE

(71) Applicant: LSI Solutions, Inc., Victor, NY (US)

(72) Inventors: Jude S. Sauer, Pittsford, NY (US); Benjamin James Boseck, Canandaigua, NY (US)

(21) Appl. No.: 19/198,914

(22) Filed: May 5, 2025

Related U.S. Application Data
(60) Provisional application No. 63/642,647, filed on May 3, 2024.
(60) Provisional application No. 63/644,732, filed on May 9, 2024.

(51) Int. Cl. A61M 60/81; A61M 60/178; A61M 60/247; A61M 60/424

(52) U.S. Cl. CPC A61M 60/81; A61M 60/178; A61M 60/247; A61M 60/424

(57) ABSTRACT

A selective blocking door device includes a coupling assembly that is configured to be removably secured to a mating base that is secured to a human heart. A frame assembly is secured to the coupling assembly, and a closure assembly is configured to linearly displace within opposing lateral slots of the frame assembly from a first open position to a second closed position. In the open position, no portion of the closure assembly is configured to be aligned with a central aperture of the mating base such that the central aperture is clear of any obstruction by the closure assembly. In the closed position, a portion of the closure assembly is configured to be aligned with the central aperture of the mating base such that the central aperture is covered and obstructed by a portion of the closure assembly.

### CROSS-REFERENCE TO RELATED APPLICATIONS

This application claims priority to U.S. Provisional Application No. 63/644,732, filed on May 9, 2024, and U.S. Provisional Application No. 63/642,647, filed on May 3, 2024, the contents of each of which is herein incorporated by reference in its entirety.

### FIELD

The claimed invention relates to a medical device, and more specifically to a selective blocking door device.

### BACKGROUND

In some procedures, such as during off-pump, beating heart LVAD (Left Ventricular Assist Device) implantation, an inlet or outlet nozzle 30 of a heart pump 28 (illustrated in FIG. 17) may be inserted into a chamber of the heart, and the heart pump 28 may pump blood into (or out of) the chamber. The heart pump 28 is typically sold or provided with a mating base 12 that is configured to be coupled to the heart 29, as illustrated in FIG. 33. Typically, the mating base 12 has an annular shape with a cylindrical member 39 having a central aperture 38 that is positioned on (and coupled to) a desired portion of the heart, and a portion of the mating base 12 is configured to releasably mate with a portion of the heart pump 28 and/or with a portion of the outlet nozzle 30 of the heart pump 28. In such an embodiment, an aperture is cored in the heart that is aligned with the central aperture 38 of the mating base 12. A distal portion of the outlet nozzle 30 is subsequently inserted through the central aperture 38 and into (or adjacent to) the aperture of the heart, at which time a portion of the outlet nozzle 30 mates with a corresponding portion of the mating base 12 such that (a) outlet nozzle 30 is sealingly coupled to the mating base 12 and (b) the distal end of the outlet nozzle 30 is in fluid communication with the chamber of the heart. Coupled as described, the pumping operation may begin and blood may be pumped into the chamber of the heart. In some embodiments, the outlet nozzle 30 may instead be an inlet nozzle, and blood can be pumped from the chamber of the heart. In this disclosure, the terms “outlet” is understood to also mean “inlet” if blood is to be pumped from the chamber.

Turning back to the procedure, and immediately after the aperture of the heart is cored, blood from the chamber of the heart, under high pressure, exits the aperture, causing significant blood loss in the patient and obscuring the treatment area so the surgeon has difficulty seeing the outlet nozzle 30 and the mating base 12, making mating more difficult, which, in turn, may result in further blood loss in the patient. In extreme cases, such severe blood loss could be fatal. Thus, it would be desirable to have a device that could reduce the amount of blood flow that follows the coring of the aperture in the heart and continues until the heart pump 28 is properly secured to the mating base 12.

### BRIEF DESCRIPTION OF THE DRAWINGS

FIGS. 1 to 2, 10 to 12, 22 to 25, and 28 to 32 illustrate various views of an embodiment of a selective blocking door device;

FIGS. 3, 4, and 5 illustrate various exploded views of a mating base, heart pump, and the embodiment of the selective blocking door device of FIG. 1;

FIGS. 6 to 9, 13 to 15, 18, 19 illustrates various views of a further embodiment of a selective blocking door device;

FIG. 16 illustrates an exploded view of a mating base, heart pump, and the embodiment of the selective blocking door device of FIG. 6;

FIG. 17 illustrates a coring guide;

FIGS. 18 to 21 illustrate various views of the selective blocking door device of FIG. 6 with the coring guide;

FIG. 26 illustrates the selective blocking door device of FIG. 6 with the mating base;

FIG. 27 illustrates an exploded view of the selective blocking door device of FIG. 1;

FIG. 33 illustrates a human heart with the mating base attached.

### DETAILED DESCRIPTION OF THE INVENTION

FIG. 16 illustrates an embodiment of a selective blocking door device 10 that is configured to removably couple to a portion of the mating base 12 during the installation or removal of the heart pump 28, and the device 10 may be removed prior to the operation of the heart pump 28. In particular, the device 10 includes a coupling assembly 14 that is configured to releasably couple to a coupling portion 40 of the mating base 12. As illustrated in FIG. 23, the coupling assembly 14 may include an elongated first coupling member 14a that extends from a first end 44a to a second end 46a along a first axis 42a, and the first axis 42a may be cambered or have the shape of an arc, which may be a segment of a circle. The coupling assembly 14 may include an elongated second coupling member 14b that extends from a first end 44b to a second end 46b along a second axis 42b, and the second axis 42b may be cambered or have the shape of an arc, which may be a segment of a circle, and the first axis 42a may have the same or substantially the same shape as the second axis 42b. The second end 46b of the second coupling member 14b may be pivotably coupled with the second end 46a of the first coupling member 14a. In some embodiments, the second end 46b of the second coupling member 14b may be directly pivotably coupled with the second end 46a of the first coupling member 14a about a hinge 13 or the second end 46b of the second coupling member 14b may be coupled to a component having a hinge 13 and the second end 46a of the first coupling member 14a may be coupled to the component having the hinge 13. The first end 42b of the second coupling member 14b may be releasably coupled with the first end of the first coupling member 14a when the device is coupled to the coupling portion 40 of the mating base 12.

In particular, in a first open position, illustrated in FIG. 23, the second coupling member 14b may be pivoted away from the first coupling member 14a about the hinge 13, and the coupling portion 14 may be positioned around a circumference of the coupling portion 40 of the mating base 12. The coupling portion 40 may include a circumferential lip 49, illustrated in FIG. 16, that extends radially outward from a distal end of the cylindrical member 39.

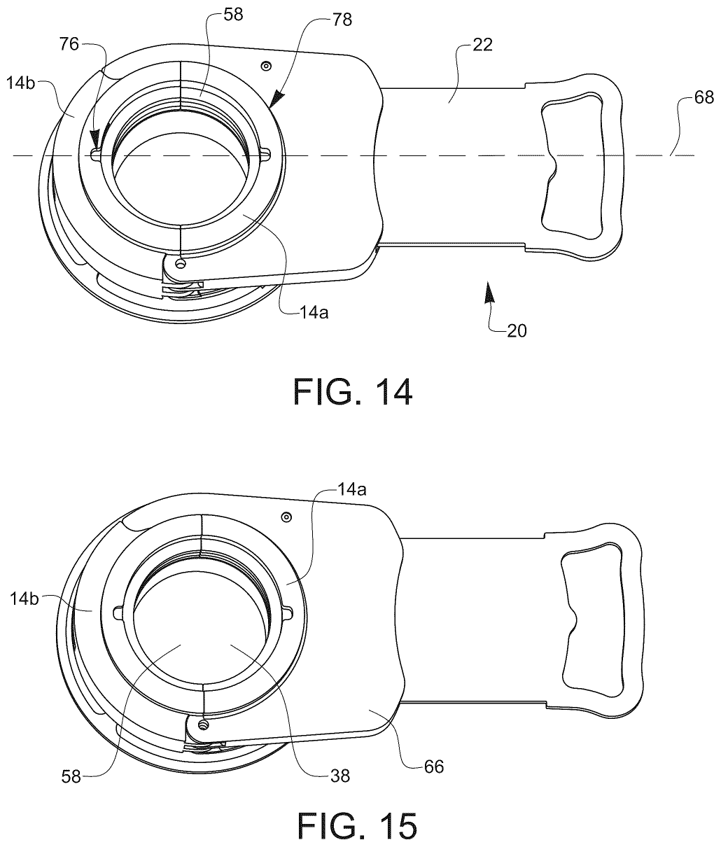

When positioned around the coupling portion 40 of the mating base 12, the first ends 44a, 44b of the first and second coupling members 14a, 14b may be pivoted into a closed position illustrated in FIG. 14, at which point the first ends 44a, 44b of the first and second coupling members 14a, 14b may be adjacent. A tab portion 47 may extend from, or be a portion of or formed at or adjacent to, the first end 44a, 44b of one of the first or second coupling members 14a, 14b, and the tab portion 47 may be received into a corresponding slot or recess 48 formed at or adjacent to first end 44a, 44b of the other of the first or second coupling members 14a, 14b to improved engagement. The tab portion 47 may be a portion of the second coupling member 14b that extends from the first end 44b towards the second end 46b of the second coupling member 14b, and the tab 47 may allow the coupling member 14 to avoid contact with a portion of the mating base 12 and/or the heart pump 28 when installing or removing the coupling member 14 from the mating base 12.

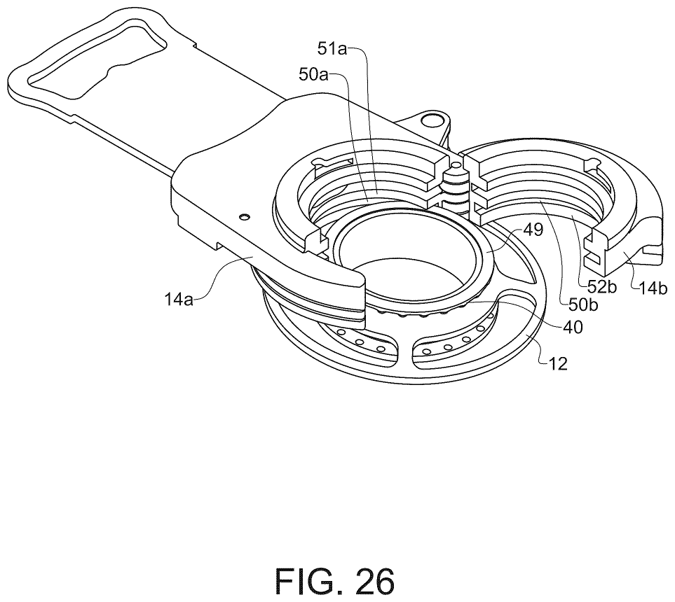

To secure the coupling portion 40 of the mating base 12 to the coupling assembly 14, a first portion of the lip 49 may be received into a first slot 50a formed along one or more inward surfaces 51a of the first coupling member 14a, as illustrated in FIG. 26, with the first and second coupling members 14a, 14b in the open position. The first and second coupling members 14a, 14b may then be pivoted into the closed position (shown in FIG. 16), such that a second portion of the lip 49 may be received into a second slot 50b formed along one or more inward surfaces 51b of the second coupling member 14a, which are illustrated in FIG. 26.

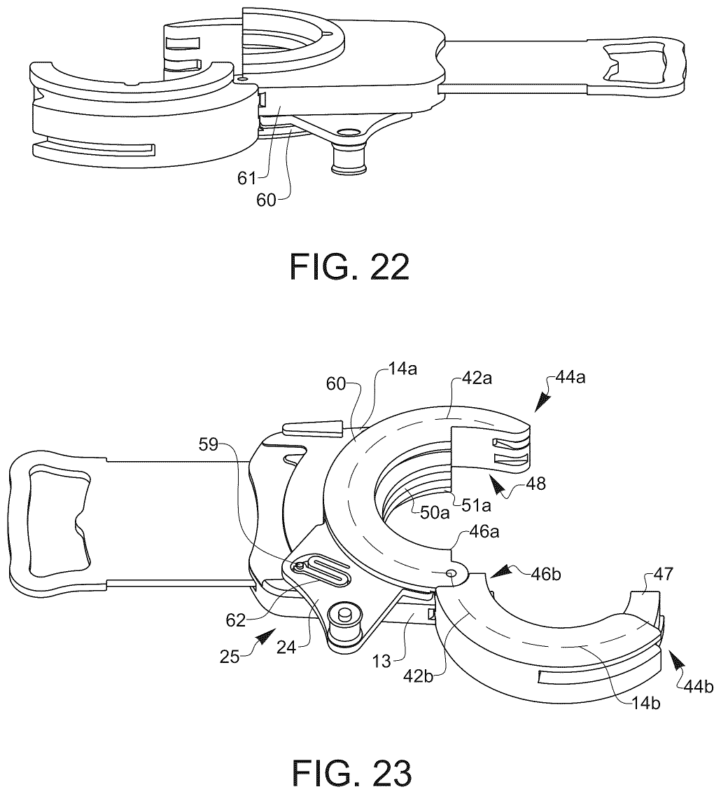

Referring again to FIG. 23, the coupling assembly 14 may further include a lever assembly 25 that may include a locking lever 24. The locking lever 24 may be planar or substantially planar, and as illustrated in FIG. 22, may extend through a slot 61 formed in one or more outward surfaces 60 of the first coupling member 14a. Referring again to FIG. 23, a latch 59 may be formed on a portion of the locking lever 24, and the latch may be disposed at an end portion of a cantilevered spring 62 that may be integrally formed along a portion of the locking lever 24.

With reference to FIG. 25, the lever assembly 25 may also include an elongated locking member 26 that may be coupled to or integrally formed with a portion of the locking lever 24. The locking member 26 may extend from a first end (not shown) to a second end 54 along a locking axis 56, with the first end being coupled to or integrally formed with a portion of the locking lever 24. The locking axis 56 may be cambered or arced such that it has the shape of a segment of a circle, and the locking axis 56 may have the same diameter as (or a slightly greater diameter than, such as 10% to 20% greater) the first axis 42a and second axis 42b of the first and second coupling members 14a, 14b, respectively. In some embodiments, an edge portion (not shown) of the locking lever 24 may be coextensive with an inner edge portion 63 of the locking member 26, and the edge portion (not shown) of the locking lever 24 may have the shape of a segment of a circle that may have a radius equal to that of the inner edge portion 63 of the locking member 26.

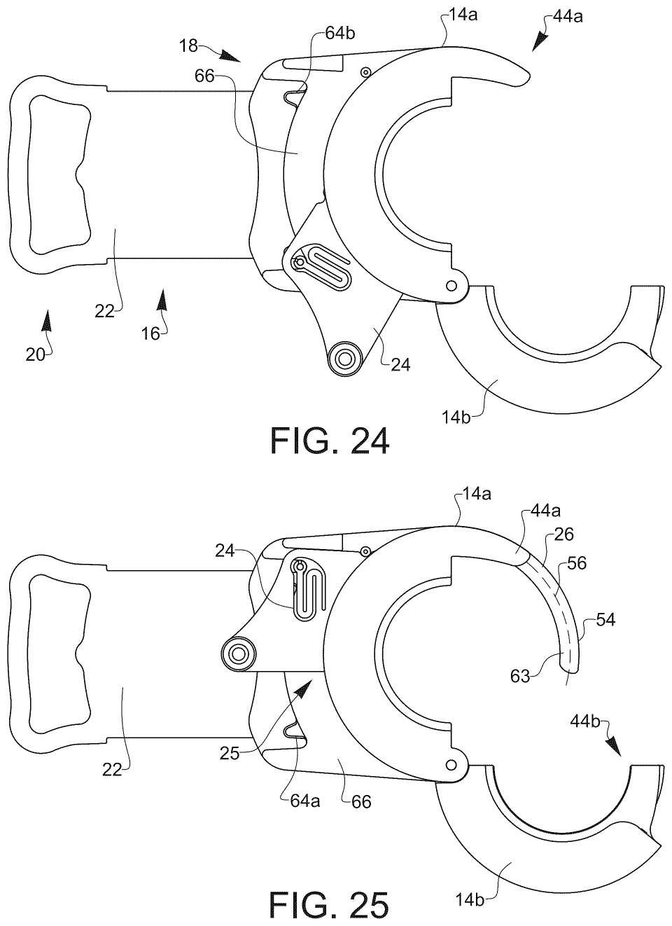

The locking member 26 may be disposed in a slot formed in a portion of the first coupling member 14a and a portion of the second coupling member 14b, and the locking member 26 may be able to rotate within the slot. When the lever assembly 25 is an unlocked position, as illustrated in FIG. 24, the first end 54 of the locking member does not extend beyond (e.g., is proximal to) the first end 44a of the first coupling member 14a.

When the locking lever 24 is rotated from the unlocked position of FIGS. 23 and 24 (in which the lever assembly 25 is in the unlocked position) towards the locked position of FIGS. 25 and 14 (in which the lever assembly 25 is in the locked position) by a user, the first end 54 of the locking member 26 extends beyond (e.g., is distal to) the first end 44a of the first coupling member 14a (along the path of the locking axis 56) and extends into the portion of the slot of the second coupling member 14b to secure the first coupling member 14a to the second coupling member 14b, thereby preventing the removal of the coupling assembly 14 from the coupling portion 40 of the mating base 12.

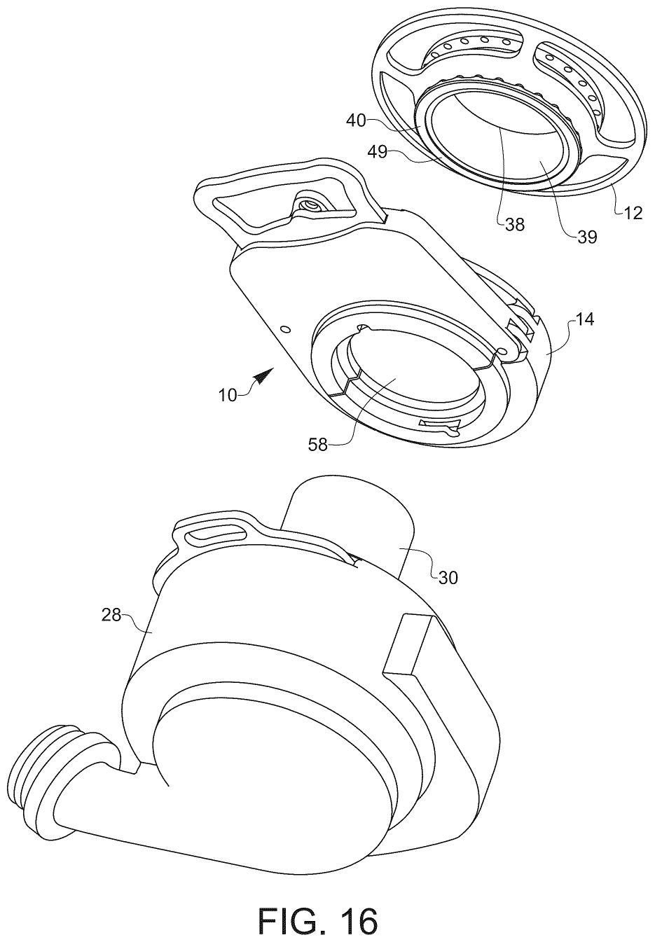

With the coupling assembly 14 in the locked position and the lever assembly 25 in the locked position, the inward surfaces 51a, 51b of each of the first and second coupling members 14a, 14b at least partially define an aperture 58 (see FIG. 15) that is coaxially aligned with the central aperture 38 of the mating base 12, and the diameter of the aperture 58 may be approximately equal to the central aperture 38 of the mating base 12.

With reference to FIGS. 24 and 25, and in the unlocked position of the lever assembly 25, the latch 59 of the locking lever 24 may engage a first mating feature 64a, such as a first protrusion that is received into a recess (not shown) formed in an underside of the latch 59. The first mating feature 64a may be formed on a portion of the first coupling member 14a or at a first portion of an extension portion 66 that extends from a portion of the first coupling member 14a. In the unlocked position of the lever assembly 25, the latch 59 of the locking lever 24 may engage a second mating feature 64b, such as a second protrusion that is received into the recess (not shown) of the latch 59. The second mating feature 64b may be formed on a portion of the first coupling member 14a or at a second portion of the extension portion 66.

As illustrated in FIG. 24, the device 10 may also have a door assembly 16 that is movably coupled to the coupling assembly 14. The door assembly 16 may include a frame assembly 18 and a closure assembly 20 that may be movably coupled to the frame assembly 18. In some embodiments, the frame assembly 18 may be formed on all or a portion of one of the first and second coupling members 14a, 14b, such as the first coupling member 14a, and the frame assembly may be formed along all or a portion of the extension portion 66. In some embodiments, the frame assembly 18 may include one or more lateral slots (not shown) that may be formed along opposing lateral portions of the extension portion 66, and the slots may be defined by one or more interior surfaces of the extension portion 66. Each of the slots may be parallel to a door axis 68, which is illustrated in FIG. 19, and the door axis may be parallel to the X-axis of the reference coordinate system of FIG. 19.

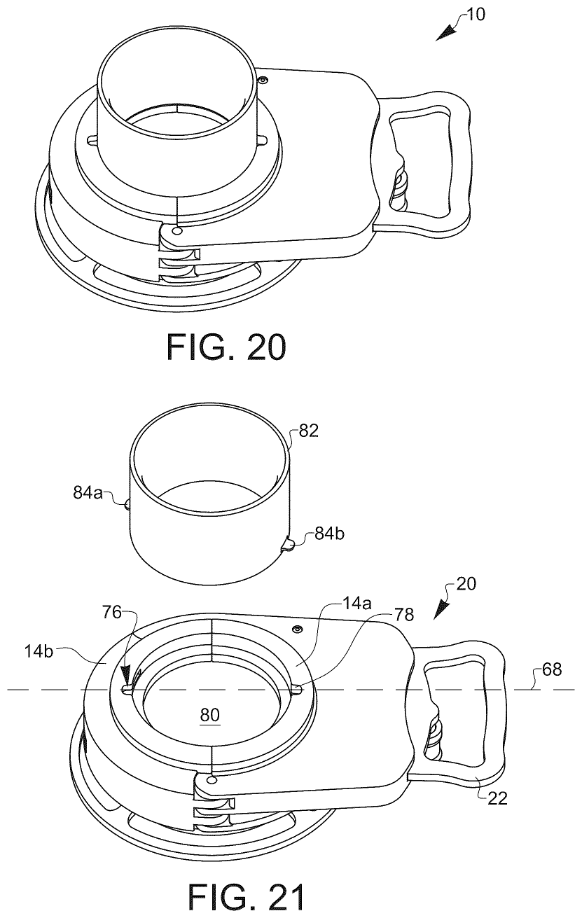

In some embodiments, the closure assembly 20 includes a planar door 22 that is configured to linearly displace within the frame assembly 18 relative to the coupling assembly 14 from a first open position to a second closed position. The door 22 extends from a first end 72 (illustrated in FIG. 9) to a second end 74 along the door axis 68, and the door 22 is configured to displace along the door axis 68 from an open position (illustrated in FIGS. 18 to 19 and 23 to 25) to a closed position (illustrated in FIGS. 20 to 21). The door 22 may include opposing lateral edge portions 70a, 70b that each extend from the first end 72 to the second end 74 and each of the opposing lateral edge portions 70a, 70b may be linear and may extend parallel or generally parallel to the door axis 68. Each of the opposing lateral edge portions 70a, 70b may be configured to be received into a corresponding one of the slots formed along opposing lateral portions of the extension portion 66 such that the door 22 may displace linearly along the door axis 68.

In the first open position, no portion of the closure assembly 20 is aligned with the central aperture 38 of the mating base 12 such that the central aperture 38 is clear of any obstruction by the door 22. In the closed position, a portion of the closure assembly 20 is aligned with the central aperture 38 of the mating base 12 such that the central aperture 38 is covered and obstructed by a portion of the closure assembly 20.

In the embodiment of FIG. 14, when the closure assembly 20 is in the first open position, the first end 72 of the door 22 is proximal to a first point 78 of the first coupling member 14a that intersects the inward surface 51a of the first coupling member 14a and the door axis 68. Thus, no portion of the first end 72 of the door 22 extends into the aperture 58 defined by the first and second coupling members 14a, 14b. In the embodiment of FIG. 21, when the closure assembly 20 is in the second closed position, the first end 72 of the door 22 is distal to a second point 76 of the second coupling member 14b that intersects the inward surface 51b of the second coupling member 14b and the door axis 68. Thus, a portion 80 of door 22 adjacent to the first end 72 of the door 22 extends across the entire aperture 58 defined by the first and second coupling members 14a, 14b.

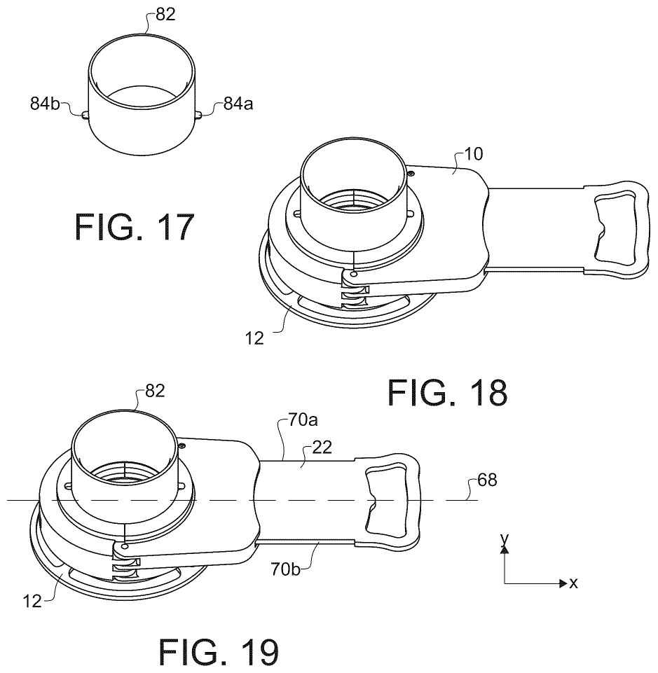

In a procedure, when the aperture is to be cored in the heart, with the closure assembly 20 is in the first open position, the surgeon places or couples a cylindrical coring guide 82 (see FIG. 19) to the coupling assembly 14 such that the bore of the coring guide 82 is aligned with the aperture 58 of the coupling assembly 14. The coring guide 82 may have an outer surface having a diameter that is slightly smaller than the diameter of the aperture 58. Each of two protrusions 84a, 84b (illustrated in FIGS. 17 and 21) may be inserted into receiving slots (not shown) formed in each of the first and second coupling members 14a, 14b and the coring guide 82 may be rotated about its longitudinal axis to secure the coring guide 82 to the coupling assembly 14.

During a procedure, and with the device 10 secured to the mating base 12 as previously described, the heart may be cored through the coring guide 82 using a coring tool in a known manner, and before a distal end of the coring tool is completely removed from the coring guide 82, the closure assembly 20 (e.g., the door 22) is displaced or transitioned into the closed position to limit or prevent blood from exiting the central aperture 38 of the mating base 12. While the closure assembly 20 (e.g., the door 22) is in the closed position, the outlet nozzle 30 of the heart pump 28 may then be secured to the mating base 12, and the closure assembly 20 (e.g., the door 22) is displaced or transitioned into the open position so blood can flow from (or to) the outlet nozzle 30 into (or from) the chamber. The coupling assembly 14 is then moved to the open position and the device 10 is removed from the mating base 12.

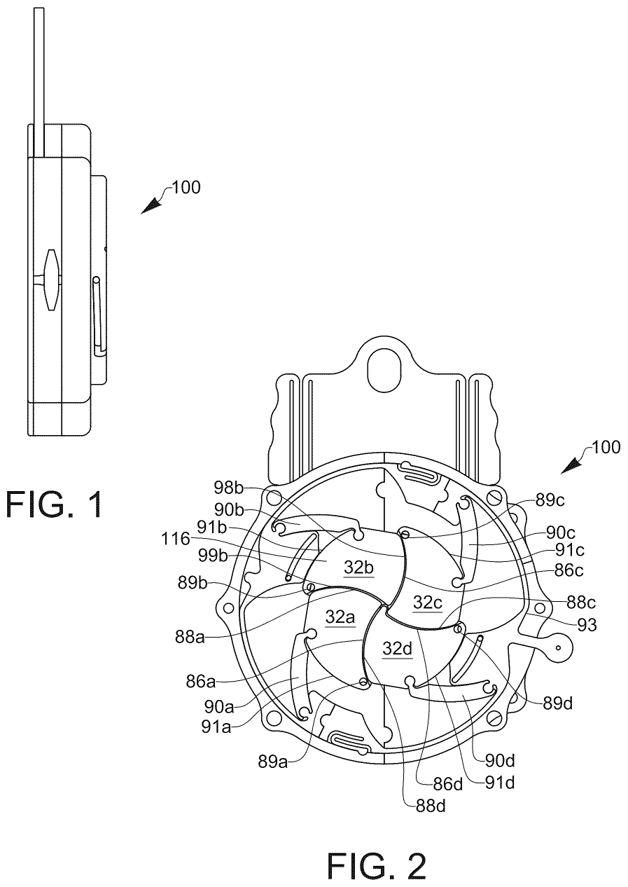

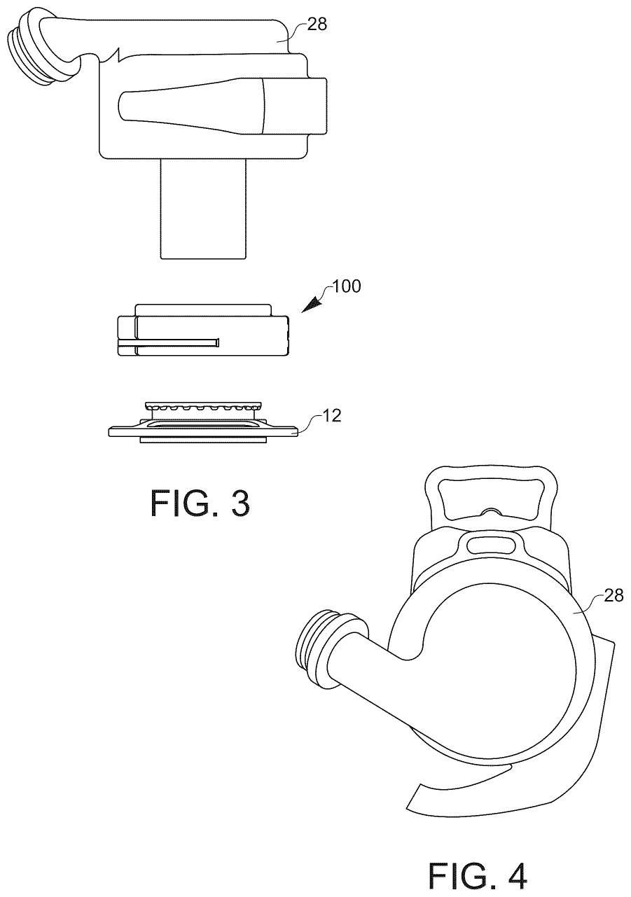

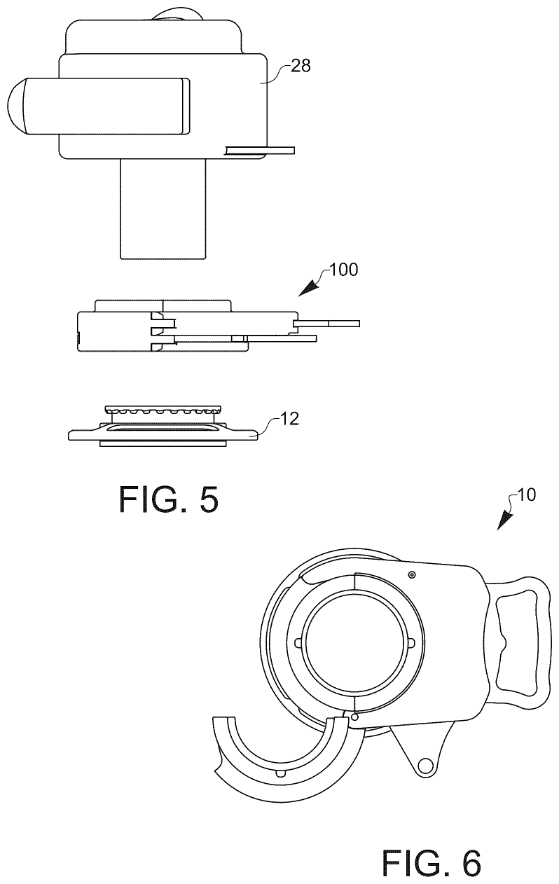

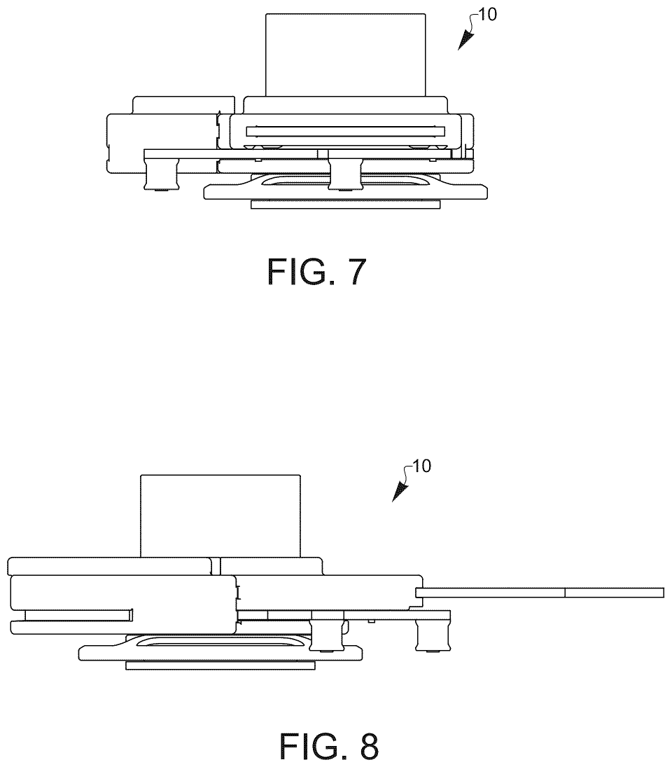

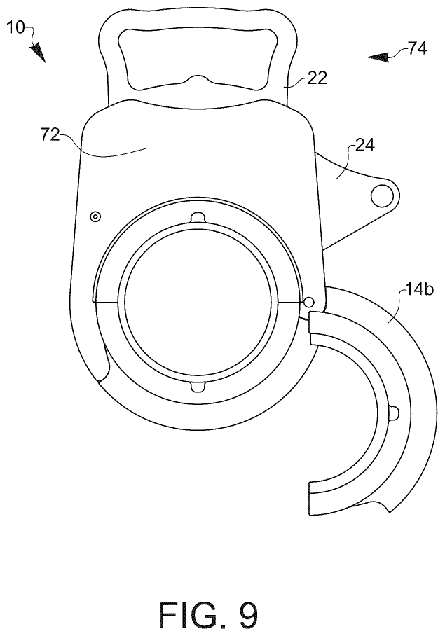

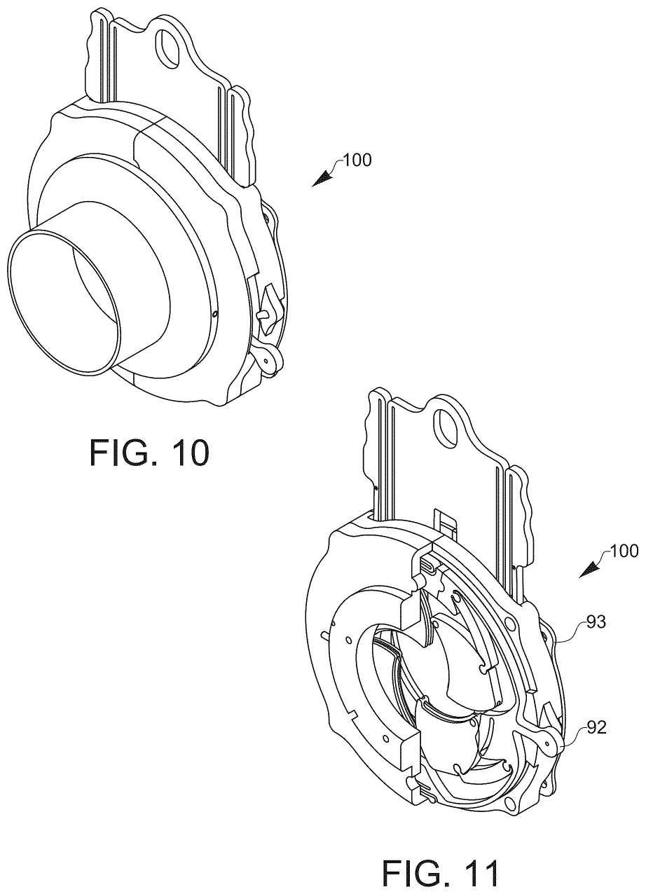

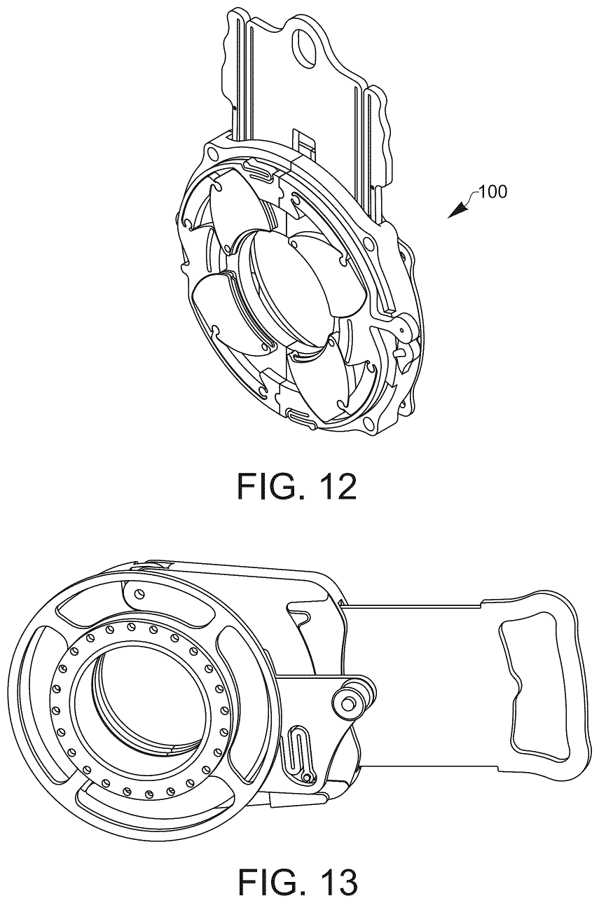

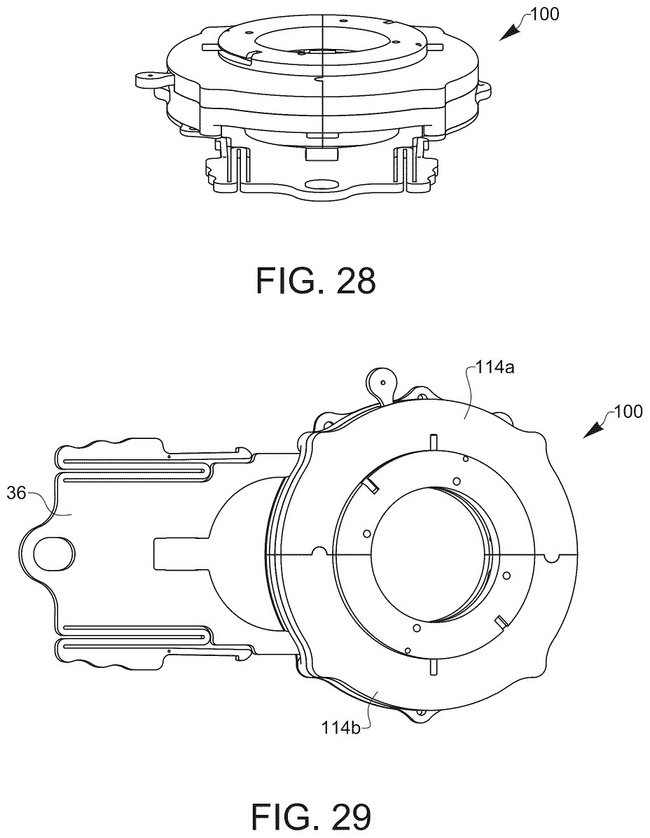

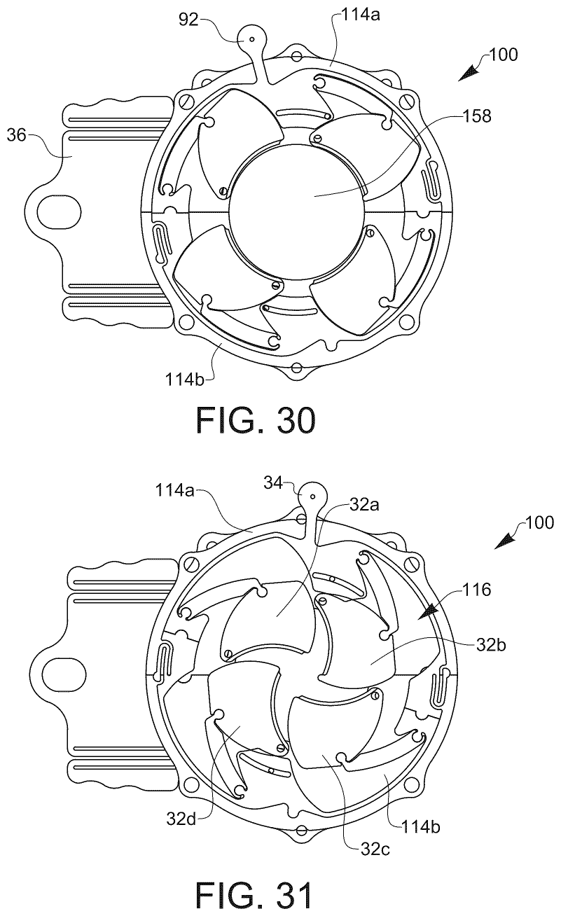

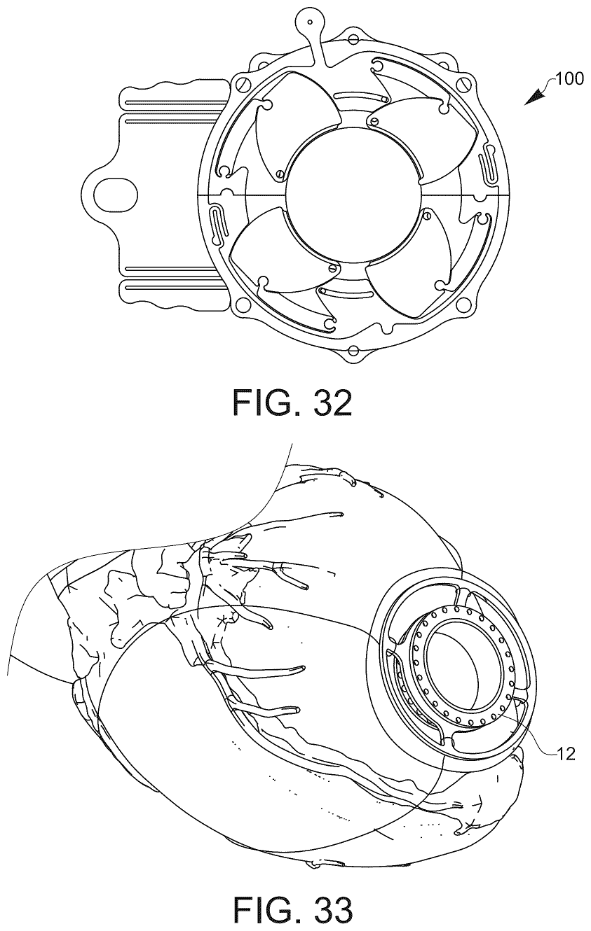

An alternative embodiment of the device 100 with an alternative door assembly 116 is illustrated in FIGS. 1 to 5, 10 to 12, and 27 to 32. In this embodiment, the door assembly 116 does not include a single planar door 22 that translates linearly, but rather a plurality (e.g., two to six) of slides 32, and, for example, may have four slides, a first slide 32a, a second slide 32b, a third slide 32c, and a fourth slide 32d. Each of the slides may be displaced between an open position of the door assembly 116 (illustrated in FIG. 30) and a closed position of the door assembly 116 (illustrated in FIG. 2) by one or more levers 90, as illustrated in FIG. 2.

These slides 32a, 32b, 32c, 32d may be actuated like the shutter mechanism of a camera. In particular, each slide 32 has a first leading edge 86 and a second leading edge, and thus the first slide 32a has a first leading edge 86a and a second leading edge 88a, the second slide 32b has a first leading edge 86b and a second leading edge 88b, the third slide 32c has a first leading edge 86c and a second leading edge 88c, and the fourth slide 32d has a first leading edge 86d and a second leading edge 88d. A first end of the first leading edge 86 contacts a first end of the second leading edge 88 of each slide 32, and an acute angle is formed between the first leading edge 86 and the second leading edge 88. However, each of the first leading edge 86 and the second leading edge 88 may not be linear, but may instead be a segment of a circle, such that when the door assembly 116 is in the open position, the first leading edges 86 of each of the slides 32 form a circular shape that may have a diameter equal to or slightly greater than the diameter of the aperture 158 defined by the first and second coupling members 114a, 114b.

Further, when the door assembly 116 is in the closed position, the first leading edge 86 of each of the slides 32 is immediately adjacent to or in contact with the second leading edge 88 of an adjacent slide 32. For example, when the door assembly 116 in the closed position of FIG. 2, the first leading edge 86a of the first slide 32a is immediately adjacent to or in contact with the second leading edge 88d of the fourth slide 32d, and the second leading edge 88a of the first slide 32a is immediately adjacent to or in contact with the first leading edge 86b of the second slide 32b.

Each of the slides 32 may also have a base edge 91 that extends from a second end of the first leading edge 86 to a second end of the second leading edge 88. For example, the first slide 32a may have a base edge 91a that extends from a second end of the first leading edge 86a to a second end of the second leading edge 88a, the second slide 32b may have a base edge 91b that extends from a second end of the first leading edge 86b to a second end of the second leading edge 88b, the third slide 32c may have a base edge 91c that extends from a second end of the first leading edge 86c to a second end of the second leading edge 88c, and the fourth slide 32d may have a base edge 91d that extends from a second end of the first leading edge 86d to a second end of the second leading edge 88d. For each slide 32, a pivot pin 89 may be disposed at a point at or adjacent to the intersection of the base edge 91 and the first edge 86 such that the slide 32 pivots about the respective pivot pin 89 from an open position (corresponding to the open position of the door assembly 116) to a closed position (corresponding to the closed position of the door assembly 116).

Each of the plurality of levers 90 (e.g., four levers 90a, 90b, 90c, 90d) may be elongated and may extend from a first end to a second end. The first end of each lever 90 may be pivotably coupled to a portion of a ring 93, and the second end of each lever 90 may be pivotably coupled with a corresponding one of the base edges 91 of the corresponding slide 32. For example, the first end of the first lever 90a may be pivotably coupled to a first portion of the ring 93, and the second end of the first lever 90a may be coupled to a portion of the first base edge 91a of the first slide 32a. Each lever 90 may have a first lateral edge (and/or a first lateral surface partially defined by the first lateral edge) that may be in contact with or adjacent to a corresponding edge (or surface partially defined by the edge) of the ring 93 when the lever is in a first position that correspond to the open position of the door assembly 116. For example, the first lever 90 may have a first lateral edge (and/or a first lateral surface partially defined by the first lateral edge) that may be in contact with or adjacent to a first corresponding edge (or surface partially defined by the edge) of the ring 93 when the first lever 90 is in a first position that correspond to the open position of the door assembly 116. The first end of each lever 90 may have a recess that receives a protrusion on the ring 93 to pivotably coupled the first end of the lever 90 to the portion of the ring 93. The second end of each lever 90 may have a protrusion that is received in a recess formed on or along the base edge 91 of the corresponding slide 32 to pivotably coupled the second end of the lever 90 to the portion of the corresponding slide 32.

As such, with the door assembly 116 in the open position, a user may displace a post 92 (see FIG. 11) coupled to the ring 93 to rotate the ring 93 in a first direction, by which each lever 90 is pivoted about the first end of the lever 90 such that the second end of each lever 90 rotates towards the center of the ring 93 (e.g., towards the aperture 58). Because the second end of each lever 90 is coupled to the base edge 91 of the corresponding slide 32, the slide 32 pivots about the corresponding pivot pin 89 from the open position, to an intermediate position (illustrated in FIG. 31), to the closed position, which corresponds to the closed position of the door assembly. For example, with the door assembly 116 in the open position, a user may displace the post 92 (see FIG. 11) coupled to the ring 93 to rotate the ring 93 in the first direction, by which the first lever 90a is pivoted about the first end of the first lever 90a such that the second end of the first lever 90a rotates towards the center of the ring 93 (e.g., towards the aperture 58). Because the second end of the first lever 90a is coupled to the base edge 91a of the first slide 32a, the first slide 32a pivots about the corresponding first pivot pin 89a from the open position, to the intermediate position and finally to the closed position, which corresponds to the closed position of the door assembly 116.

To displace the door assembly 116 from the closed position to the open position, the process is reversed. In particular, with the door assembly 116 in the closed position, the user may displace the post 92 coupled to the ring 93 to rotate the ring 93 in a second direction that is opposite to the first direction, by which each lever 90 is pivoted about the first end of the lever 90 such that the second end of each lever 90 rotates away from the center of the ring 93 (e.g., towards the corresponding coupling assembly 14a, 14b). Because the second end of each lever 90 is coupled to the base edge 91 of the corresponding slide 32, the slide 32 pivots about the corresponding pivot pin 89 from the closed position, to the intermediate position, to the open position, which corresponds to the open position of the door assembly 116. For example, with the door assembly 116 in the closed position, a user may displace the post 92 coupled to the ring 93 to rotate the ring 93 in the second direction, by which the first lever 90a is pivoted about the first end of the first lever 90a such that the second end of the first lever 90a rotates away from the center of the ring 93 (e.g., towards the corresponding coupling assembly 14a, 14b). Because the second end of the first lever 90a is coupled to the base edge 91a of the first slide 32a, the first slide 32a pivots about the corresponding first pivot pin 89a from the closed position, to the intermediate position and finally to the open position, which corresponds to the open position of the door assembly 116.

In this embodiment of the device 100, the first and second coupling members 114a, 114b may not be coupled by a hinge 22, but may be separate pieces. The first and second coupling members 114a, 114b may be coupled to the mating base 12 in substantially the same manner as the first and second coupling members 14a, 14b of the device 10.

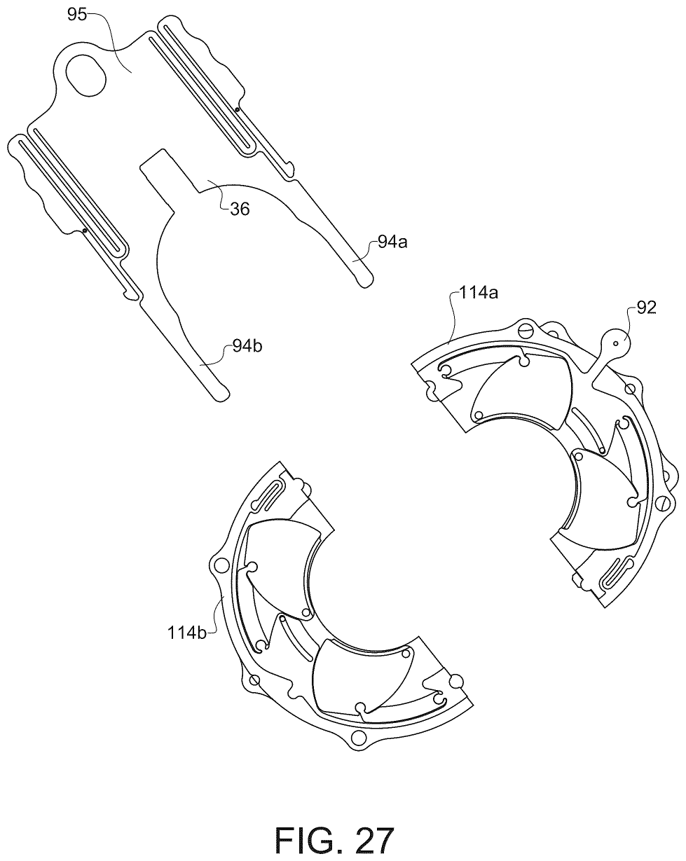

That is, to secure the coupling portion 40 of the mating base 12 to the coupling assembly 114, a first portion of the lip 49 of the cylindrical member 39 may be received into a slot or recess of the first coupling member 114a, and a second portion of the lip 49 of the cylindrical member 39 may be received into a slot or recess of the second coupling member 114b. So positioned, a clip 36 (see FIG. 27) is then inserted into corresponding slots of each of the first and second coupling members 14a, 14b, as illustrated in FIG. 29. In particular, as illustrated in FIG. 27, the clip 36 may have a first arm 94a and a second arm 94b that extends from opposed lateral edges of a body portion 95. Each of the first and second arms 94a, 94b are received in corresponding slots or recesses of the first coupling assembly 114a and second coupling assembly 114b, respectively. An inward facing edge of the body portion 95 that extends between the first arm 94a and the second arm 94b may be contoured to match a corresponding portion of the aperture 58 of the first and second coupling members 114a, 114b when the pin is fully inserted. When the distal ends of each of the first and second arms 94a, 94b are received in corresponding slots or recesses of the first coupling assembly 114a and second coupling assembly 114b, and the clip 36 is pushed towards the first coupling assembly 114a and second coupling assembly 114b to fully insert the clip 36 and secure the device 100 to the mating base 12. To remove the device 100 from the mating base 12, the process is reversed. That is, the clip 36 is moved away from the first and second coupling members 14a, 14b until the distal ends of each of the first and second arms 94a, 94b are external to the corresponding slots or recesses of the first coupling assembly 114a and second coupling assembly 114b. The first coupling assembly 114a and second coupling assembly 114b may then each be removed from the cylindrical member 39 of the mating base 12 when desired. The assembly of the device 100 during a procedure is otherwise identical to the assembly of the device 100 previously described.

Various advantages of the device have been discussed above. Embodiments discussed herein have been described by way of example in this specification. It will be apparent to those skilled in the art that the foregoing detailed disclosure is intended to be presented by way of example only, and is not limiting. Various alterations, improvements, and modifications will occur and are intended to those skilled in the art, though not expressly stated herein. These alterations, improvements, and modifications are intended to be suggested hereby, and are within the spirit and the scope of the claimed invention. The drawings included herein are not necessarily drawn to scale. Additionally, the recited order of processing elements or sequences, or the use of numbers, letters, or other designations therefore, is not intended to limit the claims to any order, except as may be specified in the claims. Accordingly, the invention is limited only by the following claims and equivalents thereto.

What is claimed is:

1. A selective blocking door device, comprising:
a coupling assembly comprising:
an elongated first coupling member extending from a first end to a second end along a first axis, wherein the first axis has the shape of an arc;
an elongated second coupling member extending from a first end to a second end along a second axis, wherein the second axis has the shape of an arc, and wherein the second end of the second coupling member is pivotably coupled with the second end of the first coupling member such that the coupling assembly is configured to be pivoted from an open position to a closed position in which the first end of the second coupling member is disposed at or adjacent to the first end of the first coupling member,
wherein each of the first coupling member and the second coupling member include a corresponding slot that is configured to receive a portion of a circumferential lip of a mating portion of a mating base that is secured to a portion of a human heart such that when the coupling assembly is in the closed position, the coupling assembly is secured to the mating portion of the mating base, and wherein in the closed position, one or more inward surfaces of each of the first and second coupling members at least partially define an aperture that is configured to be coaxially aligned with a central aperture of the mating base; and
a door assembly comprising:
a frame assembly including an extension portion, and opposing lateral slots are defined by one or more interior surfaces of the extension portion; and
a closure assembly that is configured to linearly displace within the opposing lateral slots of the frame assembly along a door axis from a first open position to a second closed position, wherein in the first open position, no portion of the closure assembly is configured to be aligned with the central aperture of the mating base such that the central aperture is clear of any obstruction by the closure assembly, and in the closed position, a portion of the closure assembly is configured to be aligned with the central aperture of the mating base such that the central aperture is covered and obstructed by a portion of the closure assembly,
wherein when the closure assembly is in the closed position, an outlet nozzle of a heart pump is configured to be secured to the mating base 12, and the closure assembly is configured to be displaced into the open position so blood can flow from the outlet nozzle of the heat pump in a chamber of the heart.

2. The selective blocking door device of claim 1, wherein the first coupling member includes a first slot formed along the one or more inward surfaces of the first coupling member and the second coupling member includes a second slot formed along the one or more inward surfaces of the first coupling member, wherein the first slot is configured to receive a first portion of the circumferential lip of the mating portion of the mating base and the second slot is configured to receive a second portion of the circumferential lip of the mating portion of the mating base.

3. The selective blocking door device of claim 1, wherein the second end of the second coupling member is pivotably coupled with the second end of the first coupling member by a hinge.

4. The selective blocking door device of claim 1, wherein the closure assembly includes a planar door that extends from a first end to a second end along the door axis, the door including opposing lateral edge portions that each extend from the first end to the second end and each of the opposing lateral edge portions are linear and extend parallel to the door axis.
